(19) United States
(12) Patent Application Publication
(10) Pub. No.: US 2019/0010533 A1
(43) Pub. Date: Jan. 10, 2019

(54) METHODS FOR SCREENING AND SELECTING TARGET AGENTS FROM MOLECULAR DATABASES

(71) Applicant: The Methodist Hospital System, Houston, TX (US)

(72) Inventor: Stephen T.C. Wong, Missouri City, TX (US)

(21) Appl. No.: 16/000,344

(22) Filed: Jun. 5, 2018

Related U.S. Application Data
(60) Provisional application No. 62/515,165, filed on Jun. 5, 2017.

(51) Int. Cl. C12Q 1/02; C12Q 1/6876; G06F 19/28

(52) U.S. Cl. CPC C12Q 1/025; C12Q 1/6876; C12Q 2600/158; C12Q 2600/136; G06F 19/28

(57) ABSTRACT

The present disclosure relates to methods for screening for a modulator of a target protein. The present disclosure further relates to a systematic disease drug repositioning (SMART) method which integrates experimental and computational biology methods systematically with public transcriptomic profile data to enable fast-track identification and confirmation of novel drug candidates.

### CROSS REFERENCE TO RELATED APPLICATIONS

This application claims the benefit of U.S. Provisional Patent Application Ser. No. 62/515,165 filed Jun. 5, 2017, which are expressly incorporated herein by reference in their entirety.

### FIELD

The present disclosure relates to methods for screening for a modulator of a target protein. The present disclosure further relates to a systematic disease drug repositioning (SMART) method which integrates experimental and computational biology methods systematically with public transcriptomic profile data to enable fast-track identification and confirmation of novel drug candidates.

### BACKGROUND

Alzheimer's disease (AD) currently afflicts 5.3 million people in the United States alone. Despite many years of research, outside of symptomatic treatment, no clear therapeutic options are available for Alzheimer's disease (AD) patients. Conventional drug discovery paradigms to identify new therapeutic candidates are ill-equipped to combat a disease as complex as AD. What is needed are new drug discovery paradigms and methods for screening and selecting promising drug candidates using the large amounts of public transcriptomic profile data.

The methods disclosed herein address these and other needs.

### SUMMARY

Disclosed herein are methods for screening for a modulator of a target protein. In addition, a systematic disease drug repositioning (SMART) framework is disclosed herein which integrates experimental and computational biology methods systematically with public transcriptomic profile data to enable fast-track identification and confirmation of novel drug candidates.

In one aspect, disclosed herein is a method for screening for a modulator of a target protein, comprising:

contacting a cell with at least one primary candidate agent;

identifying the at least one primary candidate agent that modulates the target protein;

obtaining publicly available large transcriptomic profiles of cellular responses to the at least one primary candidate agent;

performing a first iteration to extract gene expression signatures for the at least one primary candidate agent;

ranking all secondary candidate agents from the publicly available large transcriptomic profiles of cellular responses based on a similarity score of the transcriptomic profile to the at least one primary candidate agent;

selecting the modulator of a target protein from the secondary candidate agents when the similarity score is above a determined threshold.

In one embodiment, the target protein is tau. In one embodiment, the modulators affect tau phosphorylation. In one embodiment, the similarity score of the transcriptomic profiles is measured by a cMAP algorithm (or some other ranking scheme).

In one embodiment, additional iterations are performed, wherein the modulator of a target protein is added back to the list of primary candidate agents, and new modulators of the target protein are obtained by repeating the screening process.

In some embodiments, the gene expression signatures include whole genome transcriptomic profiles. In some embodiments, the gene expression signatures include transcriptomic profiles for selected gene sets.

In another aspect, disclosed herein is a computer implemented method of selecting viable target agents having a predicted drug interaction response in a patient, the method comprising:

a computer processor connected to computerized memory storing computer implemented instructions configured to iteratively repeat the following steps until converging on a final set of viable target agents:
retrieving search results from a database stored in the memory and accessible by the processor, wherein said search results identify a first set of primary candidate agents;
ranking the primary candidate agents in the first set according to pre-established criteria stored in the memory;
storing in the memory a search set of molecular traits for a selected set of laboratory validated agents selected from the ranked primary candidate agents;
using the search set of molecular traits to search the database for additional sets of secondary candidate agents exhibiting the molecular traits.

In some embodiments, the molecular traits comprise a molecular signature, a transcriptomic profile, and/or a phenotypical response. In some embodiments, the computer implemented instructions are further configured to modulate the molecular signature data in the search set to tune the search set to a preferred phenotype.

In an additional aspect, disclosed herein is a computer implemented method of identifying a set of target agents capable of completing selected biochemical tasks in a drug interaction process, the method comprising:

a computer processor connected to computerized memory storing computer implemented instructions configured to iteratively repeat the following steps until converging on a final set of viable target agents;

performing an electronic search of at least one database stored in the memory and accessible by the processor, wherein said search results identify a set of primary candidate agents;

extracting a signature for a target phenotype from each of said primary candidate agents;

compiling an expression profile in regard to the target phenotype for each of primary candidate agents;

ranking the primary candidate agents in the set according to pre-established criteria stored in the memory;

storing in the memory a search set of molecular traits for a selected set of laboratory validated agents selected from the ranked primary candidate agents;

refining respective signatures for a target phenotype in regard to the laboratory validated agents and creating an updated search set of molecular traits; and

using the search set of molecular traits to search the database for additional sets of primary target agent candidates exhibiting the molecular traits.

In some embodiments, extracting the signatures comprises transforming transcriptomic data for the primary candidate agents into a series of enrichment scores. In some embodiments, the enrichment scores comprise compressed representations of the transcriptomic data.

In some embodiments, the ranking comprises summarizing the expression signatures and comparing to control conditions. In some embodiments, the ranking comprises generating a combined score incorporating similarities between perturbation profiles and chemical properties for each primary candidate agent and comparing the combined score.

### BRIEF DESCRIPTION OF THE DRAWINGS

The accompanying figures, which are incorporated in and constitute a part of this specification, illustrate several aspects described below.

FIG. 1. The workflow of the SMART framework for drug repositioning and discovery.

FIG. 2. Pilot SMART screen used 20 primary hits to identify 5 new compound hits that inhibit pTau (phospho-Tau or phosphorylated Tau). These hits were validated using the AD-in-a-dish model, and almost completely inhibited Tau phosphorylation.

FIG. 3. Graph theory analysis showing relationships among target signatures, predicted hit candidates, and validated hits. (Left) 17 primary hits (blue) predicted 85 candidate compounds. Five (yellow) almost completely inhibit pTau in validation studies while another 5 (green) partially inhibit pTau; (Right) degree-sorted version of the connected sub-graph in (A) reveals that 4 of 5 yellow nodes have a degree larger than 4, which ranked among top 18 of all 85 predicted compounds in degree of a node.

FIG. 4. Ivermectin and its 16 predictions, which include 4 out of 5 nodes confirmed by cell based validations.

FIG. 5. The structure for the proposed deep belief network implemented in the SMART framework.

FIG. 6. Time-lapse synaptogenesis assay identifies pre-synaptic hyperactivity caused by thiorphan's treatments. (A, B) Control conditions before and after de-staining, with upward arrows indicating active boutons while downward arrows indicating inactive boutons; (C, D) FM dye uptake under control or thiorphan treatments; (E) Automatic image quantification revealed pre-synaptic hyperactivity caused by thiorphan treatment.

FIG. 7. RNA-seq and canonical pathway analysis shows significant overlaps between clonal 3D AD models and human AD patient brains. a. Pearson correlations of global gene expression profile among 2D undifferentiated control ReN cells, 3D control (G2#B2on), AD #A5 (#A5, moderate Aβ42/40 ratio ˜0.2), AD #D4 (#D4, high Aβ42/40 ratio, ˜1.4), and AD #H10 (#H10, extra high Aβ42/40 ratio, ˜1.7). Units are log CPM. b. Volcano plots show −log₁₀(FDR) vs log FC distribution for G2#B2 (control) vs AD #A5 (AD), AD #A5 DMSO vs AD #A5 BSI (BACE inhibitor, Ly2886721), and AD #A5 DMSO vs AD #A5 GSM (gamma secretase modulator, SGSM15606) transcriptomic signatures. Significantly differentially expressed genes in blue=log FC <−1.0, FDR <0.05 red=log FC >1.0, FDR <0.05. c. Canonical pathway analysis between G2#B2 and AD #A5 (Ingenuity pathway analysis, Qiagen). d. Analysis of common canonical pathways. The pathway analysis among G2#B2 vs AD #A5, AD #A5 DMSO vs AD #A5 BSI, and AD #A5 DMSO vs AD #A5 GSM. Activation z-scores indicate that majority of decreased pathways in AD #A5 are restored by BSI and/or GSM treatments. e. Comparison of enriched pathways between the 3D G2#B2 vs AD #A5 and normal brains vs AD patient brains (from the publicly available datasets). The analysis showed many common pathways significantly decreased both in human AD brains and the 3D AD #A5 samples.

FIG. 8. Validating the impact of primary hit candidates using multiple human AD cell lines with different Aβ42/40 ratios. Control and AD cells were differentiated for 6 weeks in 3D culture conditions with drug treatments in last 3 weeks. Levels of insoluble p-tau (pThr181tau) and total tau were measured by Mesoscale ELISA while actin and Tuj1(neural marker) were measured by quantitative dot blot analyses with LiCor infrared laser system. p-Tau levels were normalized either by Tuj1 or total tau. Relative decreases of phospho tau levels in each experiment (n=4 to 5) were color-coded and scored.

FIG. 9. Validation of primary hit candidates. Primary hit candidates were confirmed using Western blot analysis (a) and quantitative immunofluorescence staining in 3D AD models with high Aβ42/40 ratios (#HReN and #A4H1) (b). PHF1 pSer396/Ser404 tau antibody was used to detect changes in phospho tau in 3D AD #HReN cells treated with DMSO vehicle, ebselen, or leflunomide.

FIG. 10A-10B. Systematic modeling of RNAseq data reveals shared changes for two screening hits. (a) PPI networks involving APP, MAPT as well as 15 down-regulated (dark grey: IFNA1, IFNA2, TLR7, IRF3, IFNAR1, TLR9, IL1B, IFNG, TNF, TGM2, MAP3K7, ZAP70, EIF2AK2, IL29, PRL) and 7 up-regulated (light grey: SOCS1, EGF, IFIH1, IL1RN, BTK, GAPDH, MAPK1) genes after separate treatments of ebselen or leflunomide. Red edges illustrate PPI connecting APP to members of a group of 7 significantly changed genes. PPI information was extracted from STRING database version 10.5 with the cutoff for confidence score at 0.4. (b) A sub-network involving 12 genes and 6 pathways are significantly down-regulated (dark grey nodes with log FC<−1.5) by the treatments of candidates ebselen and leflunomide.

### DETAILED DESCRIPTION

Disclosed herein are methods for screening for a modulator of a target protein. In addition, a systematic disease drug repositioning (SMART) framework is disclosed herein which integrates experimental and computational biology methods systematically with public transcriptomic profile data to enable fast-track identification and confirmation of novel drug candidates.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

As used in the specification and claims, the singular form “a,” “an,” and “the” include plural references unless the context clearly dictates otherwise. For example, the term “a cell” includes a plurality of cells, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “candidate agent” refers to any molecule to be tested in the provided methods to determine whether the candidate agent modulates the target protein. Candidate agents can include small molecules or biomolecules. Small molecule candidate agents encompass numerous chemical classes, though typically they are organic molecules. Biomolecule candidate agents include, but are not limited to, peptides/proteins, saccharides, fatty acids, steroids, purines, pyrimidines, or antibodies (or fragments thereof) or derivatives, structural analogs or combinations thereof.

As used herein, the term “subject” or “host” or “patient” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human. In some embodiments, the pharmacokinetic profiles of the systems of the present invention are similar for male and female subjects.

Methods—SysteMAtic drug ReposiTioning and discovery (SMART)

In one aspect, disclosed herein is a method for screening for a modulator of a target protein, comprising:

contacting a cell with at least one primary candidate agent;

identifying the at least one primary candidate agent that modulates the target protein;

obtaining publicly available large transcriptomic profiles of cellular responses to the at least one primary candidate agent;

performing a first iteration to extract gene expression signatures for the at least one primary candidate agent;

ranking all secondary candidate agents from the publicly available large transcriptomic profiles of cellular responses based on a similarity score of the transcriptomic profile to the at least one primary candidate agent;

selecting the modulator of a target protein from the secondary candidate agents when the similarity score is above a determined threshold.

In one embodiment, the target protein is tau. In one embodiment, the modulators affect tau phosphorylation. In one embodiment, the similarity score of the transcriptomic profiles is measured by a cMAP algorithm (or some other ranking scheme).

In one embodiment, additional iterations are performed, wherein the modulator of a target protein is added back to the list of primary candidate agents, and new modulators of the target protein are obtained by repeating the screening process.

In some embodiments, the gene expression signatures include whole genome transcriptomic profiles. In some embodiments, the gene expression signatures include transcriptomic profiles for selected gene sets.

In another aspect, disclosed herein is a computer implemented method of selecting viable target agents having a predicted drug interaction response in a patient, the method comprising: a computer processor connected to computerized memory storing computer implemented instructions configured to iteratively repeat the following steps until converging on a final set of viable target agents:
retrieving search results from a database stored in the memory and accessible by the processor, wherein said search results identify a first set of primary candidate agents;
ranking the primary candidate agents in the first set according to pre-established criteria stored in the memory;
storing in the memory a search set of molecular traits for a selected set of laboratory validated agents selected from the ranked primary candidate agents;
using the search set of molecular traits to search the database for additional sets of secondary candidate agents exhibiting the molecular traits.

In some embodiments, the molecular traits comprise a molecular signature, a transcriptomic profile, and/or a phenotypical response. In some embodiments, the computer implemented instructions are further configured to modulate the molecular signature data in the search set to tune the search set to a preferred phenotype.

In an additional aspect, disclosed herein is a computer implemented method of identifying a set of target agents capable of completing selected biochemical tasks in a drug interaction process, the method comprising:

a computer processor connected to computerized memory storing computer implemented instructions configured to iteratively repeat the following steps until converging on a final set of viable target agents;

performing an electronic search of at least one database stored in the memory and accessible by the processor, wherein said search results identify a set of primary candidate agents;

extracting a signature for a target phenotype from each of said primary candidate agents;

compiling an expression profile in regard to the target phenotype for each of primary candidate agents;

ranking the primary candidate agents in the set according to pre-established criteria stored in the memory;

storing in the memory a search set of molecular traits for a selected set of laboratory validated agents selected from the ranked primary candidate agents;

refining respective signatures for a target phenotype in regard to the laboratory validated agents and creating an updated search set of molecular traits; and

using the search set of molecular traits to search the database for additional sets of primary target agent candidates exhibiting the molecular traits.

In some embodiments, extracting the signatures comprises transforming transcriptomic data for the primary candidate agents into a series of enrichment scores. In some embodiments, the enrichment scores comprise compressed representations of the transcriptomic data.

In some embodiments, the ranking comprises summarizing the expression signatures and comparing to control conditions. In some embodiments, the ranking comprises generating a combined score incorporating similarities between perturbation profiles and chemical properties for each primary candidate agent and comparing the combined score.

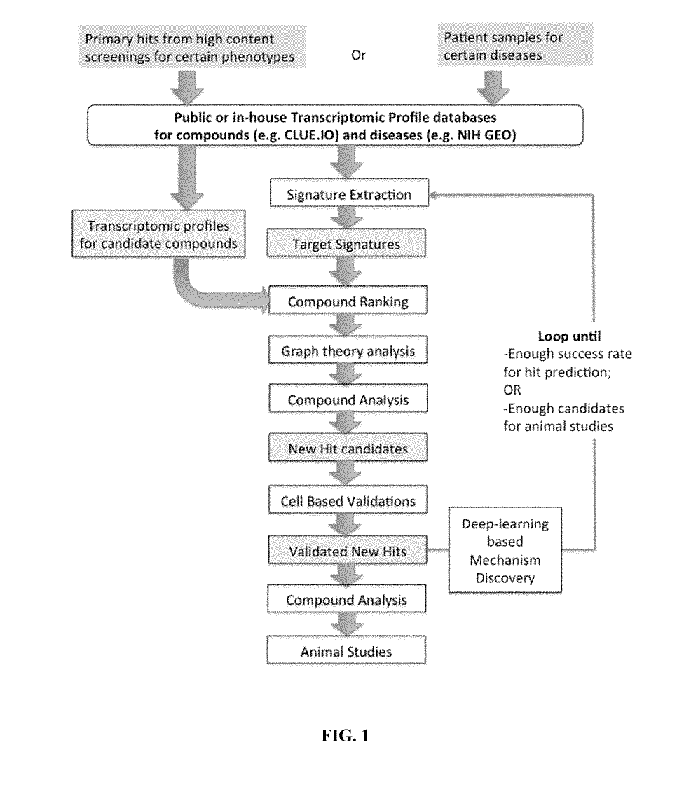

Disclosed herein is an integrative screening and deep learning framework to enable fast, systematic drug repositioning and discovery (see FIG. 1). This bioinformatics-driven iterative workflow can be used to predict optimal known drugs or small molecule compounds for certain biochemical tasks, either mimicking the transcriptomic changes corresponding to certain desirable phenotypes; or reversing the pathway activities underlying disease related phenotypes. Such a prediction is achieved by leveraging large (publicly available or in-house proprietary) transcriptomic profiles regarding subjects with various diseases as well as those recording cellular responses to various perturbations, especially small molecular compound treatments. These I/O and analytic strategies ensure that public or in-house transcriptomic profiles generated using different technologies and platforms, e.g., RNAseq and microarray, are seamlessly incorporated. The resolution for each specific biochemical task revolves around a panel of “target transcriptomic signatures” which were extracted from subjects with target phenotypes, i.e. a panel of screening hits or a group of patients with certain disease phenotype. The signature extraction step serves as the interface for accepting feedback information flow and initiating new loops. IN some embodiments, the first iteration starts with signatures covering the whole genome, and the results undergo cell assay validations and expand the training sets of desired phenotype vs. control for deep learning based mechanism discovery, ultimately leading to a refined signature consisting of phenotype-related pathways.

Subsequent iterations can start with a signature focused on pathway changes correlated to phenotype changes of interest, improving the identification of candidates for new hits.

Novel computational algorithms are developed for the key steps of signature extraction, compound ranking, and graph-theoretical analysis as shown in FIG. 1. The results from cell-based validation and mechanism discovery are fed back to modify the signature extraction step, with the goal of providing more accurate target signatures for compound ranking in another iteration, initiating an iterative workflow to improve the success rate for hit prediction, and expanding the group of repurposed or discovered drug candidates validated by animal studies for achieving the target phenotype.

### Signature Extraction

The signature extraction step summarizes the transcriptomic changes underlying the target phenotype, so that the expression profiles for all the candidate compounds can be compared to and ranked based on these changes. The extraction step should generate the type of signatures that can facilitate such comparisons and rankings. The ranking should be able to proceed even when the target signatures and the expression profiles for candidates were generated using different platforms or technologies.

For more robust signature extraction in the framework, Gene Set Enrichment Analysis (GSEA)^{28,31} is used to transform the transcriptomic data into a series of enrichment scores for functionally related gene sets. For the expression profile of each compound, GSEA provides enrichment scores for up to 13,000 gene sets defined in the MSigDB database²⁸. The scores from categories C2.CP (1,330 canonical pathways covering databases including KEGG^{32,33}, BIOCARTA^{34,35} and REACTOME^{36,37}), C3 (836 motif gene sets³⁸ covering targets of miRNA and transcription factors³⁹), C5 (1454 Gene Ontology^{40,41} terms covering biological process, molecular function, and cellular compartment), and H (50 hallmark gene sets defined by the MSigDB database⁴²) are used. The compound perturbation omics' signature is compressed into ˜3,620 enrichment scores. This new signature extraction scheme facilitates inclusion of transcriptomic profiles generated by other technology and platforms, as GSEA generates signatures of equal size after platform-specific processing within each dataset.

### Compound Ranking

Most available compound ranking schemes use a similar strategy as the cMAP algorithm, which summarizes the expression signature for each compound treatment using genes with the top 100 and bottom 100-fold expression changes comparing to control conditions. This scheme may be over-simplified in that it is vulnerable to expression profile outliers while the fixed cut-off number for significant genes may lead to ignorance on certain key expression changes and thus underestimation of the global picture of pathway activities.

To measure the similarity between target signatures i and candidate signature j, a combined score is generated incorporating the similarities between their perturbation profiles and chemical properties. The similarity metric⁴³ is combined with the metrics in the STITCH database⁴⁴ to quantify the similarity between two signatures i and j. After GSEA analysis, the similarity metric S_{G} (i,j) is defined as the Pearson Correlation Coefficients between the two vectors. In the case where both signatures i and j were generated from small molecule compound treatments, an additional similarity metric, Sₛ(i,j) is defined based on the STITCH database⁴⁴ by integrating a combined score of the structure similarity and text-mining similarity score. The structure similarity is defined by the Tanimoto 2D chemical similarity scores' while the text mining similarity is computed by mining a curated database, such as OMIM⁴⁶ and MEDLINE, using a co-occurrence scheme and a natural language processing approach^{47,48}. The two similarity metrics combined as: S(i,j)=αSₛ(i,j) S_{G}(i,j),j=1, 2 . . . 20,413, where a is the parameter controlling the level of emphasis for structure information. Here, each target compound i corresponds to one of 17 primary hits in the pilot run, and for each i, there are 20,413 similarity scores that can be normalized into Z-scores. Top-ranked compounds with p-value <0.05 are selected as candidate hits.

### Graph-Theoretical Analysis:

In each iteration of the screening workflow, the relationships among target signatures, predicted hit candidates, and validated hits can be modeled using a directed graph (DG) model⁴⁹. After compound ranking, each target compound i is associated with a group of predicted compounds Pᵢ={pᵢ^{(x)}}, x=1, 2 . . . m, which are selected based on the cut-off of compound similarities. A directed graph G=(V,E) can then be defined, with the set of vertices V=1∪P, where I={1, 2 . . . n} is the set of target compounds and P={P₁, P₂ . . . Pₙ} is the set of predicted compounds. In a pilot run, the set of target compounds is the group of primary hits with LINCS data; thus n=17 and the size of P is 85. Meanwhile, the set of edges, E only includes directed edges in the form of e={i,pᵢ^{(x)}}, with weight on the edge wₑ=S(i,pᵢ^{(x)}), i.e., each edge will always be from one target compound to one of its predicted compounds, with the similarity between two connected compounds serving as the edge weight.

### Iterative Running of Functions Using Feedback Information Flow:

As shown in FIG. 1, all functional modules defined above run iteratively to effectively search the space of all available compounds, find new screening hits, and ultimately provide candidates for novel therapy. Feedback information flow is used to control both the width and depth of the search scheme. Refining the number of bait compounds and modulating signature content can help control the search width. In some embodiments, given the panel of predicted compounds from any iteration, 3D-cell based validation assays assure that only true hits corresponding to significant phenotype changes serve as the “baits” for the next iteration.

Meanwhile, based on the validation results, all predicted compounds are added to the training sets of desired phenotype vs. control, allowing the deep-learning model to gain a better understanding of transcriptomic features underlying phenotype changes of interest. The output of the deep-learning analytics consists of a series of key pathway changes, which can then help refine the content of transcriptomic signatures used in the next iteration, allowing the search scheme to focus on key pathways that continuously generate validated predictions. The depth of this workflow is correlated to its efficacy; specifically the success rate of hit prediction overall and within each iteration. The iterative workflow can be terminated when enough (for example, 5-10) novel drug candidates are collected for animal studies or when the updated mechanism information brings the success rate of hit prediction to a desirable level (for example, over 75%).

### Computer Implemented Methods

In example implementations, at least some portions of the activities may be implemented in software provisioned on networking device 102. In some embodiments, one or more of these features may be implemented in hardware, provided external to these elements, or consolidated in any appropriate manner to achieve the intended functionality. The various network elements may include software (or reciprocating software) that can coordinate in order to achieve the operations as outlined herein. In still other embodiments, these elements may include any suitable algorithms, hardware, software, components, modules, interfaces, or objects that facilitate the operations thereof.

Furthermore, the network elements of FIG. 1 (e.g., network devices 102) described and shown herein (and/or their associated structures) may also include suitable interfaces for receiving, transmitting, and/or otherwise communicating data or information in a network environment. Additionally, some of the processors and memory elements associated with the various nodes may be removed, or otherwise consolidated such that single processor and a single memory element are responsible for certain activities. In a general sense, the arrangements depicted in the Figures may be more logical in their representations, whereas a physical architecture may include various permutations, combinations, and/or hybrids of these elements. It is imperative to note that countless possible networking and computing configurations can be used to achieve the operational objectives outlined here. Accordingly, the associated infrastructure has a myriad of substitute arrangements, design choices, device possibilities, hardware configurations, software implementations, equipment options, etc.

In some of example embodiments, one or more memory elements can store data used for the operations described herein. This includes the memory being able to store instructions (e.g., software, logic, code, etc.) in non-transitory media, such that the instructions are executed to carry out the activities described in this Specification. A processor can execute any type of instructions associated with the data to achieve the operations detailed herein in this Specification. In one example, processors could transform an element or an article (e.g., data) from one state or thing to another state or thing. In another example, the activities outlined herein may be implemented with fixed logic or programmable logic (e.g., software/computer instructions executed by a processor) and the elements identified herein could be some type of a programmable processor, programmable digital logic (e.g., a field programmable gate array (FPGA), an erasable programmable read only memory (EPROM), an electrically erasable programmable read only memory (EEPROM)), an ASIC that includes digital logic, software, code, electronic instructions, flash memory, optical disks, CD-ROMs, DVD ROMs, magnetic or optical cards, other types of machine-readable mediums suitable for storing electronic instructions, or any suitable combination thereof.

These devices may further keep information in any suitable type of non-transitory storage medium (e.g., random access memory (RAM), read only memory (ROM), field programmable gate array (FPGA), erasable programmable read only memory (EPROM), electrically erasable programmable ROM (EEPROM), etc.), software, hardware, or in any other suitable component, device, element, or object where appropriate and based on particular needs. Any of the memory items discussed herein should be construed as being encompassed within the broad term “memory element.” Similarly, any of the potential processing elements, modules, and machines described in this Specification should be construed as being encompassed within the broad term “processor.”

The list of network destinations can be mapped to physical network ports, virtual ports, or logical ports of the router, switches, or other network devices and, thus, the different sequences can be traversed from these physical network ports, virtual ports, or logical ports.

### EXAMPLES

The following examples are set forth below to illustrate the compounds, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Identification of Novel Drugs or Bioactive Compounds that can Inhibit Alzheimer's Disease-Related pTau Accumulation

In this example, a high-content screening (HCS) scheme used a library of ˜2,100 compounds to identify 38 primary hit compounds that can significantly inhibit the accumulation of pTau within neuron cells in 3D culture. This workflow to identify the mechanisms underlying those screening hits can be used to effectively discover more compounds that can generate similar phenotype. As a proof of concept, the transcriptomic profiles hosted by the Broad Institute's LINCSCloud data warehouse²⁸⁻³⁰ through the NIH LINCS program were used in the initial study. The LINCSCloud dataset covers ˜20 cell lines' response profile to 20,413 small molecule compounds, including ˜1,300 FDA approved drugs and more than 5,000 bioactive compounds, experimental compounds, and shelved drugs.

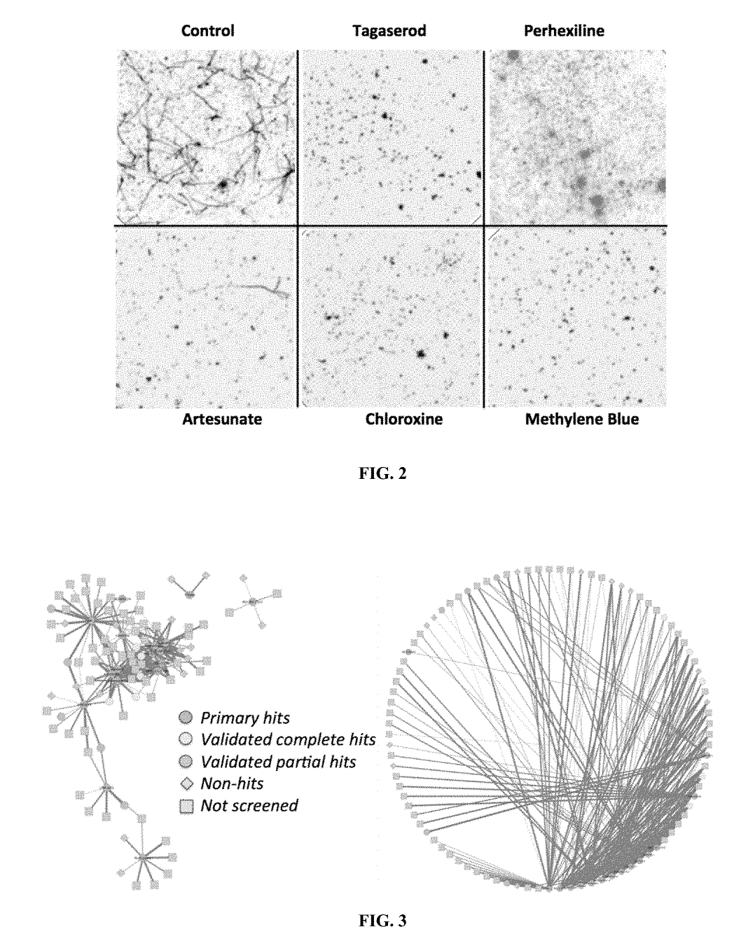

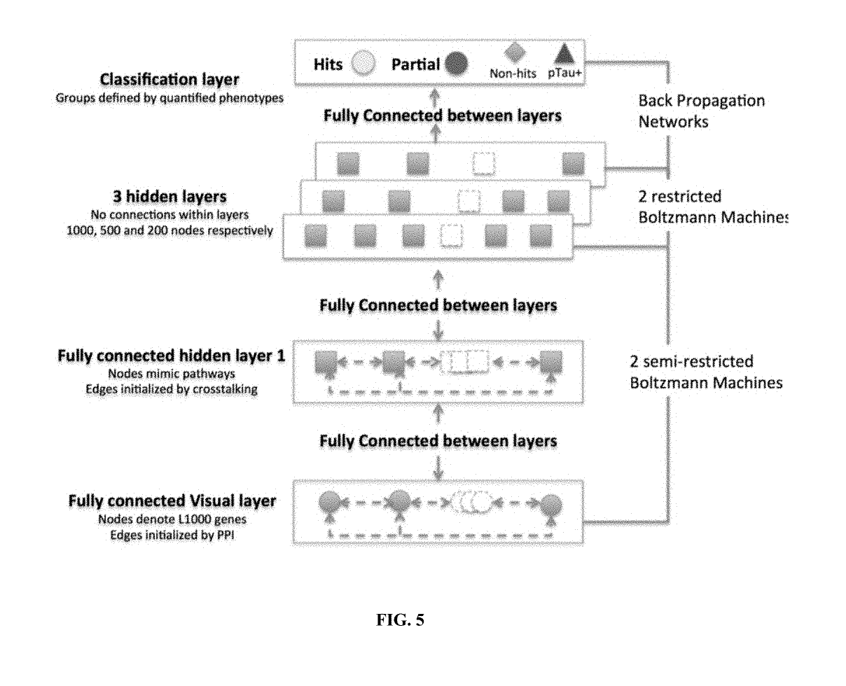

Twenty-two of the 38 aforementioned screening hits had LINCS data covering the perturbation profiles for at least 4 cell lines. From these twenty-two hits, 2 were eliminated because no known drug candidates ranked high enough based on transcriptomic similarities to these two primary hits; and 3 others were removed upon inspection of the compound properties of the predictions they made, i.e., the predicted drugs may be toxic or unfit for systematic use. Thus, the 17 primary hits were used to initiate a pilot run using the SMART framework. The cMAP algorithm⁷ was used to rank all compounds in the LINCSCloud, based on the similarity of transcriptomic profiles to each of the 17 primary hits. If any compound was determined by cMAP algorithm to have a similarity score larger than 90 to at least one of the primary hits, it was identified as a hit candidate. After filtering based on pharmacology features, 85 candidates predicted by 17 primary hits remained; 26 of these 85 compounds were purchased for validation after analysis for pharmacology and medical practice features. According to the validation results, 10 of these predictions significantly inhibited pTau (See Table 1). Five compounds almost completely inhibited pTau in the reformatted high content version of AD-in-a-dish model (with compound names listed in FIGS. 2 and 4), achieving phenotypes comparable to those from the top-3 hits (ivermectin, mg624, and pentamidine) in the primary screen.

TABLE 1
Compounds identified as candidate agents and their previously known functions

| Name | Previously known function |
|---|---|
| tegaserod maleate | to treat irritable bowel syndrome and constipation |
| perhexiline maleate | approved in Australia and New Zealand as a prophylactic antianginal |
| | agent |
| liothyronine sodium | to treat hypothyroidism and myxedema coma, also used as |
| | augmentation agent to treat major depressive disorder |
| dasatinib monohydrate | a cancer drug to treat chronic myelogenous leukemia and Philadelphia |
| | chromosome-positive acute lymphoblastic leukemia |
| pazopanib | a cancer drug to treat renal cell carcinoma and soft tissue sarcoma |
| hydrochloride | |
| vemurafenib | to treat BRAF V600E mutation positive unresectable or metastastic |
| | melanoma |
| olaparib | a cancer drug to treat ovarian, breast, and prostate cancers with |
| | hereditary BRCA1 and BRCA2 mutations |
| artesunate | an antimalarial drug |
| methylene blue | mainly used to treat methemoglobinemia, also used as a dye |
| chloroxine | an antibacterial drug to treat infectious diarrhea, intestinal microflora |
| | disorders, giardiasis, and inflammatory bowel disease |

Even without further iterations, this smart drug screening workflow achieved a 5.88% (5/85) success rate in predicting hits, more than a 51-fold improvement over the 0.114% (3/2640) hit identification rate of the primary screening.

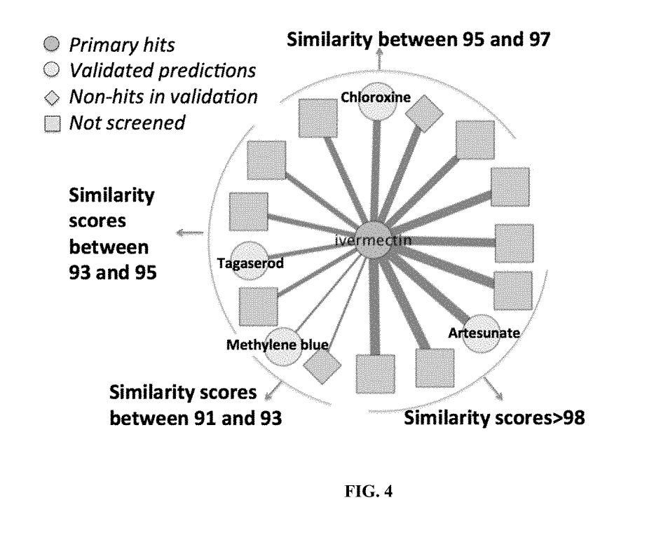

FIG. 3 summarizes the results of graph theory analysis: 17 primary hits (blue nodes) connected to 85 predicted compounds (yellow, green and gray) through a total of 215 edges, the thickness of the edge is proportional to the edge weight. Three isolated communities exist in the graph: one of the primary hits, Ro90-7501, forms one isolated community with its four predictions; another primary hit, TTNPB, forms another community with its two predictions. The remaining nodes form the largest connected community. FIG. 3 also shows that connected community in a degree-sorted circular view: a total of 94 connected nodes (15 primary hits and 79 predictions) are positioned in a circle, with the compound having the most neighbors located in the six o'clock position and all other nodes located in counter-clockwise order with descending degrees. This view reveals that 14 out of 17 primary hits have a degree larger than 7; also, 4 of 5 (yellow) validated hits have a degree larger than 4, ranking them among top 18 out of all 85 predicted compounds (chloroxine in FIG. 2 has a degree of 3 and ranked 22nd); meanwhile, all 5 (green) partial hits have a degree no more than 2.

In addition to the above “big picture” analysis of the overlap between predictions made by multiple target compounds, directed graph (DG) is also used to assess the relationships between individual target compounds and its predictions. Ivermectin has the most significant phenotype of the 38 primary hits (FIG. 4), and 4 of 5 successful predictions (except for Perhexiline in FIG. 2) in the pilot run have similarity scores larger than 90 with ivermectin. Of the 16 compounds predicted by ivermectin, 10 (gray squares) were not purchased after analyzing their previous medical usages. Thus 4 out of 6 (66.7%) ivermectin predictions tested were validated, much higher than 5.88% for the pilot run overall. By comparing with FIG. 3, Artesunate and Chloroxine have similarity scores larger than 95 in FIG. 4, yet their overall degrees are smaller than those of compounds Tegaserod and Methylene Blue.

This study revealed specific graph-theoretic characteristics for the validated hits from the pilot run. Thus, more validated hits can be revealed with more iterations of the workflow, these validated hits serve as cluster centers and divide the whole space of 20,413 compounds into highly connected clusters, and the validated hits are enriched in these compound clusters such that it is possible to predict hit compounds within certain clusters based on the graph-theoretic features, e.g. yellow nodes among the largest community in FIG. 3 mostly have larger degrees.

The graph in FIG. 3 is expanded using the nodes brought in by future iterations of the workflow. A series of graph-theoretical features, e.g., the panel of eighteen features⁵⁰, are calculated for each node. These features represent different aspects of graph-theoretical properties. Features like clustering coefficient⁵¹ and information centrality^{52,53} for each validated hit are incorporated with hierarchical clustering methods to divide the connected part of the graph into highly connected or highly centralized sub-graphs. Within each sub-graph, SVM classifiers⁵⁴⁻⁵⁶ are trained to differentiate validated hits vs. non-hit compounds based on their graph theory properties. When a new compound is introduced to the graph, it is assigned to one of the pre-defined sub-graphs based on its similarity with known hits, and its graph theory features are fed into the specific classifier for this sub-graph to generate a confidence score as to whether this compound tends to have similar graph features as those known validated hits in the same sub-graph.

### Compound Feature Analysis:

After unbiased ranking of all candidate compounds by their transcriptomic similarity to each target compound, a series of filtering procedures are applied based on the features of top-ranked compounds. First, confirmed non-hits, i.e. compounds that failed to show significant phenotypes in previous screening or validations, are eliminated. Remaining compounds are assigned into four categories: approved drugs, clinical trial drugs, investigational compounds, and compounds with limited information.

In some examples, the focus is on finding novel AD therapies, and in some examples only approved drugs (currently approved by FDA, discontinued, or internationally approved) or clinical trial drugs are kept as candidates for repurposing.

These candidates are filtered by pharmacological features and other practical considerations including toxicity (drugs requiring Health Safety Committee (HSC) review based on GHS Cat.1⁵⁷ are eliminated), systemic usage (drugs not approved for systemic usage are eliminated), and commercial availability.

Deep Belief Networks (DBN) for Identifying Mechanisms Underlying pTau Regulation:

As the iterative workflow proceeds, more compounds have matched transcriptomic and phenotypic profiles to show whether they effectively regulate pTau. A deep learning based AI model using DBN is developed to: 1) use unsupervised deep learning to understand the regulatory structure of transcriptome data, and 2) incorporate class labels defined from quantified pTau phenotypic profiles to identify gene modules underlying pTau regulation. Level-4 differential expression profiles from LINCSCloud is also used.

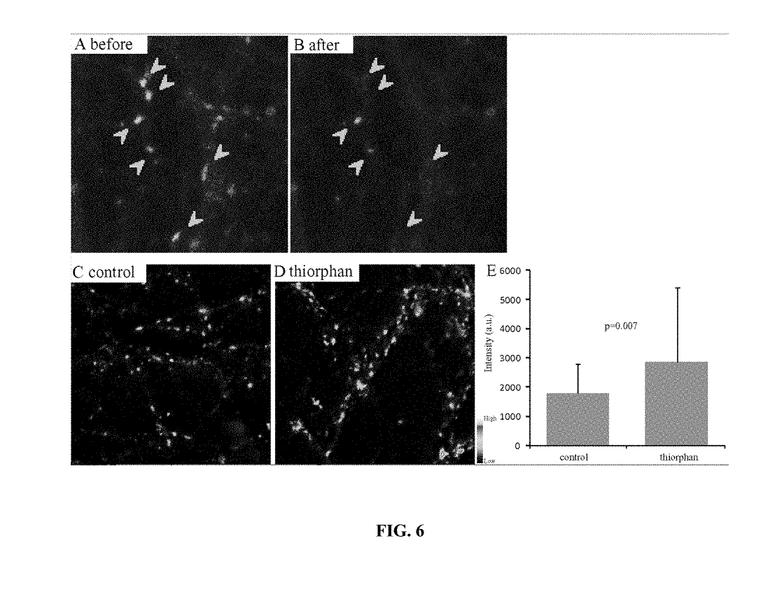

The planned DBN is a stacked neural network with six layers (FIG. 5). The bottom five layers (named overall-visible layer and hidden layers 1-4, respectively, from bottom up) accomplish the unsupervised deep learning by forming four restricted Boltzmann machines (RBM). The top layer includes group labels defined by cell-based validations, e.g. confirmed hits, partial hits, non-hits, and even increased pTau. It is used to adjust parameters in the lower levels in back propagation (top-down) style. Each node from the lowest layer corresponds to individual gene expression levels measured for each L1000 landmark gene; the nodes learned from hidden layer 1, whose values are determined jointly by nodes in the visual layer, can be interpreted as gene modules. The values of nodes in hidden layers 2-4 are determined jointly by the nodes in the immediate lower layer, and thus potentially reveal higher order regulatory and crosstalk mechanisms among gene modules.

An RBM consists of a layer of visible variables vᵢ, i=1, . . . , m, and a layer of hidden variables hᵢ,j=1, . . . , g. The nodes are fully connected across two layers, with no connection allowed within the same layer. Let symmetric metric W=(w_{i,j})_{m×g} represent weights between two layers of variables, while a=(a₁, . . . , aₘ) and b=(b₁, . . . , b_{g}) represent bias vectors corresponding to each variable in visible and hidden layers, respectively. Given a joint configuration (v, h) for the RBM, an energy function of an RBM model can be defined for binary visible and hidden unit as E(v, h; θ)=a^{T}v+b^{T}h+v^{T}Wh, with θ=(a, b, W). In this case, hidden layers 2-4 are composed of binary units while the overall visible layer consists of random variables following Gaussian distributions (because level-4 data are Z-scores), which corresponds to the expression profile of m=978 landmark genes measured in the Broad Institute L1000 protocol. For the RBM involving overall visible layer and hidden layer 1, the energy function is rewritten as:

$$E ( v , h ; θ ) = {\sum }_{i = 1}^{m} \frac{{( {v}_{i} - {a}_{i} )}^{2}}{2 {σ}_{i}^{2}} + {\sum }_{i = 1}^{m} {\sum }_{j = 1}^{g} \frac{{v}_{i}}{{σ}_{i}} {w}_{ij} {h}_{j} + {\sum }_{j = 1}^{g} \frac{{v}_{i}}{{σ}_{i}} {b}_{j} {h}_{j} .$$

Either way, the probability density function of a joint configuration (v, h) can be defined as

$$f ( v , h ; θ ) = \frac{1}{Z ( θ )} exp ( - E ( v , h ; θ ) ) ,$$

with conditional density distribution defined accordingly. Correlations among input variables are allowed as the learning procedures canceling the correlations out.⁶⁴

In this case, the overall visible layer has m=978 while hidden layer 1 is allocated 3,000 nodes, comparable to the combined number of canonical pathways (1330) and GO terms (1454) in the MSigDB database⁶⁵. W=(w_{i,j})_{m×g} between these two layers is initialized to reflect the gene set membership, i.e., w_{i,j}=1 if gene i belongs to gene set (pathway or GO term) j according to the MSigDB. This weight is bound to change according to the data structure during the learning steps, reflecting the pathway rewiring effects of gene mutations in cancer cell lines. Hidden layers 2-4 are planned to have 1,000, 500, and 200 nodes, respectively, to uncover the hierarchical structure and crosstalk among gene modules.

Currently, there are more than 1,600 compounds with matched transcriptomic profiles and phenotype labels (>50% of the 2,640 compounds in primary screening have transcriptomic profiles in LINCSCloud, and the pilot run gave phenotypic labels to 26 predicted compounds, confirming 5 as hits) that are used to learn the DBN parameters using contrastive divergence −k (CD−k) algorithms⁶⁴. Each RBN is trained greedily with the change of weight given by: Δwᵢⱼ=ε( [symbol] vᵢhᵢ [symbol] _{data}− [symbol] vᵢhᵢ [symbol] _{reconstruction}), with ε the learning rate and [symbol] vᵢhᵢ [symbol] _{data} the fraction of time the i-th visible unit and hidden unit are simultaneously on when the hidden units are driven by training data. [symbol] vᵢhᵢ [symbol] _{reconstruction} is the corresponding fraction when the hidden layers are reconstructed after k rounds of Gibbs sampling^{66,67}.

The CD-k algorithm approximates the result of maximizing the log likelihood function of the data by minimizing the Kullback-Leibler divergence and has been proven useful in many cases, even with k=1. In this example, the learning of the DBNs is carried out on the computer cluster in the Houston Methodist Hospital Data Center. Next, the results for k=1-5 are compared for their performance of differentiating different phenotype groups.

### Example 2. Identification of Novel Therapeutic Candidates Based on High-Content Screening Using iPSC Derived Parkinson's Disease Model

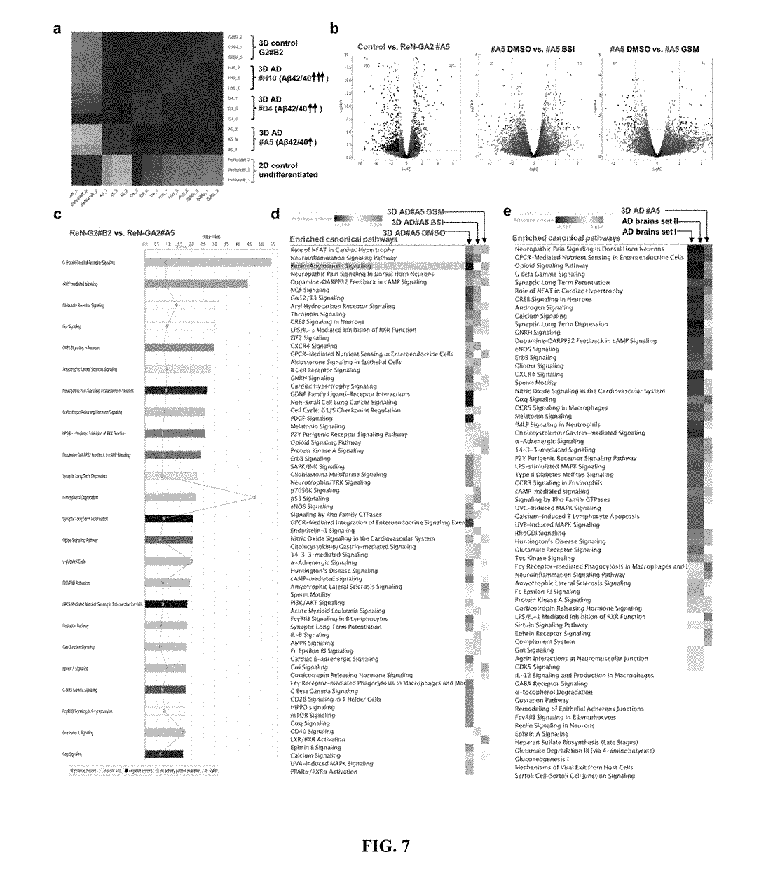

A high content screening (HCS) is carried out on existing 3,000 known drugs and compounds to systematically characterize the effects of known drugs or bioactive compounds on the Parkinson's Disease (PD) induced pluripotent stem (iPS) cell model, with the aim to identify effective hits that can be validated in PD mouse models. FIG. 6 shows an assay that was applied to primary neurons to detect compounds with effects of enhancing pre-synaptic hyperactivity. Cells were stained with FM1-43 (FIG. 6A) and de-stained by KCl stimulation (FIG. 6B). Time-lapse imaging was carried out from the dye uptake until the synapses were completely de-stained. Automatic image quantifications were used to identify compounds like thiorphan (FIG. 6 C-E), which causes pre-synaptic hyperactivity. A similar assay is applied in iPS cells as well as neuron cell models for PD. In addition to using the prevalence of synaptogenesis as the main readout for the high content screening (HCS), the heterogeneous nature of stem cell differentiation is addressed by identifying and quantifying the prevalence of novel phenotypes other than stem cell and synaptogenesis based on morphological features. Such consideration of cell population heterogeneity brings deeper insight in the HCS and can help identify potential compounds that are causing specific type of differentiations benefiting cure of PD.

To explore the molecular mechanisms underlying the phenotype of interest, i.e. synaptogenesis from both normal and PD derived iPS cell models, it is critical to connect high-content cellular phenotype profiles with the corresponding transcriptomic profiles recording pathway activities. Publicly, there are larger amount of patient- or cellular-level transcriptomic profiles generated from various technologies (e.g. microarray and RNAseq). Specifically, Broad Institute hosts a LINCSCloud data warehouse, where transcriptomic profiles is available to record ˜20 cell lines' molecular-level responses to more than 20,000 small molecular compounds²⁰⁻²². Within this data warehouse, the transcriptomic profiles for a primary iPSC-derived neural progenitor under different compound treatments are the most valuable in mechanism understanding and drug candidate prediction.

The SMART framework as shown FIG. 1 can incorporate the screening results and public available transcriptomic profiles on iPS cells, use DBN to explore the differentially expressed genes and pathways, and use such understanding of mechanisms to identify compound candidates that can generate similar pathway changes. The phenotype-genotype relationship requires the iterative design of the workflow described herein. After 3,000 known drugs were screened on a PD cell-based assay, DBN is used to classify the transcriptomic profiles of hit vs. non-hit and provide a target signature consisted of significant pathway changes. Each cycle can provide 50-100 non-screened candidates through compound ranking regarding the target signature, and the HCS setup is used to validate the effects of those candidates. The validation results would then add to the hit vs. non-hit training sets and help update the DBN model. Such iteration of “HCS-DBN-compound ranking-HCS” would lead us through the search space of over 18,000 drug compounds with LINCS data yet not included in the primary screening and is to determine the 3-4 best drug repositioning candidates ready for testing in Parkinson's Disease animal model.

### Example 3. RNA-Seq and Canonical Pathway Analysis Shows Significant Overlap Between Clonal 3D AD Models and Human AD Patient Brains

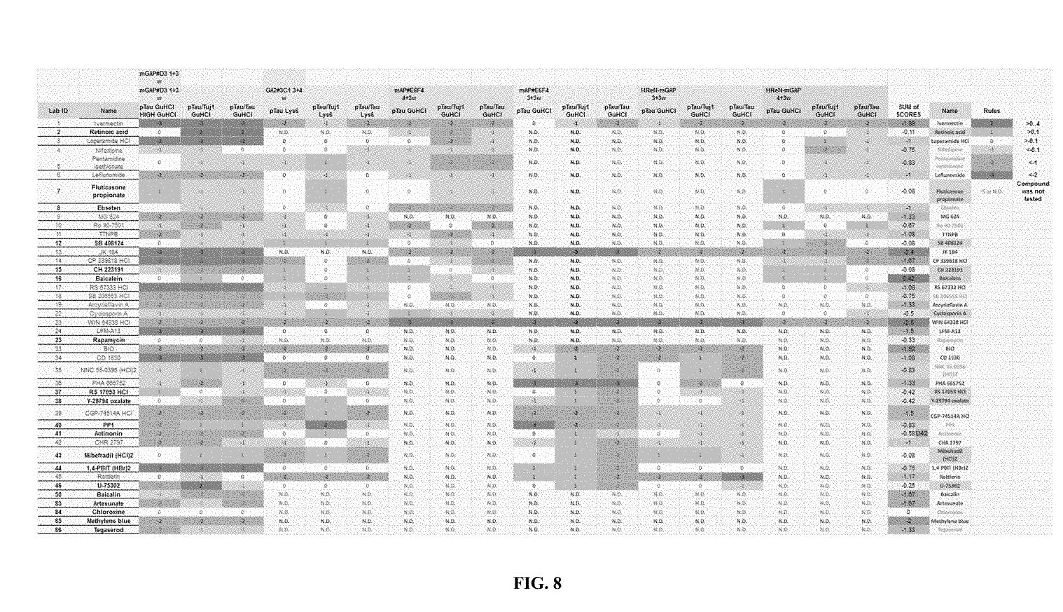

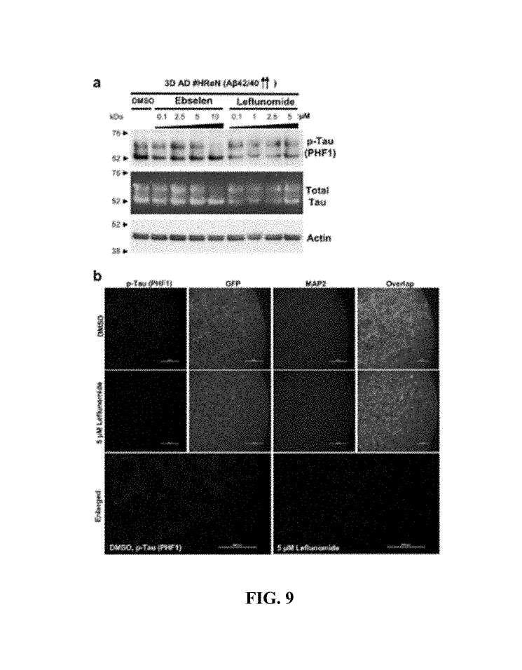

Multiple single-clonal 3D AD cell lines were used to confirm drug candidates identified from the SMART approaches. These single-clonal AD cell lines provide more reproducible results for drug screening as compared to the original mixed AD cell lines. Another advantage of using multiple single clonal lines is that the impact of candidate drugs on 3D AD models are tested with mild, moderate, or severe AD pathology. It was shown that single-clonal AD cells with higher Aβ42/40 ratio (#D4, #H10, #A4H1; FIG. 7-8) displayed robust AD pathology including pathological AP accumulation and insoluble aggregation of phospho- and total tau species (p-tau, t-tau), as compared to AD cells with lower Aβ42/40 ratio (#A5, #3C1; FIG. 7-8).

To examine the multiple single-clonal AD models, unbiased whole genome RNA-seq analyses were performed to compare gene expression profiles among the clonal AD models with different Aβ42/40 ratios, as compared to control 3D cultures and undifferentiated 2D control cells (FIG. 7a-d). It was found that clonal AD cell lines with different Aβ42/40 ratio (#D4, #H10, # showed distinctive differential gene expression patterns as compared to control 3D cells) (FIG. 7a). Differential gene expression profile of 3D AD cultures were analyzed after treating anti-Aβ drugs (BACE1 inhibitor, Ly2886721; Gamma-secretase modulator (GSM), GSM15606) (FIG. 7b). Canonical pathway analysis of differentially expressed genes between 3D control (G2#B2) and 3D AD model (#A5) showed significantly enriched pathways including glutamate receptor signaling, synaptic long term potentiation/depression, cAMP/CREB signaling, LPS/IL1 and RXR, which overlap with previously proposed AD pathogenic cascades. (FIG. 7c). Treatments with anti-AP drugs significantly altered some of these pathways (FIG. 7d). More importantly, enriched pathways were compared between the 3D AD model (#A5) and AD patient brains using available AD brain RNA-seq database.

Comparative analysis showed significant enrichment of common pathways between the 3D AD model and AD brains, including glutamate signaling, synaptic long term potentiation/depression, CREB/cAMP and Calcium signaling (FIG. 7e). These results show that this 3D AD model recapitulates AD pathogenic cascades.

### Example 4. Cross-Validation of Candidate Drugs Using Multiple Human AD Cell Lines with Different Aβ42/40 Ratios

The hit candidates from SMART screening were cross-validated. FIG. 8 is a summary showing an example of the cross-validation approach. The impact of the compounds on insoluble p-tau (pThr181tau) and total tau levels were measured by Mesoscale ELISA (n=4 to 5) and the impact levels were summarized by coding. The summary of the effects from four clonal AD cell lines with different Aβ42/40 ratios and the overall impact scores were calculated (FIG. 8). Most of the drug candidates generally decreased insoluble p-tau levels, but some of the candidates seem to alter p-tau only in select AD lines, showing these compounds work in differential action mechanisms. More importantly, most of the identified compounds decreased p-tau levels in the severe 3D AD cells with high Aβ42/40 ratio (#D4). Similar cross-validation studies were also performed with the same cells for the impact on pathogenic AP species. Some of the drugs significantly decreased AP accumulation as well as p-tau, while most of the other candidates only decreased p-tau levels (data not shown). These results show different action mechanisms of these compounds.

### Example 5. Validation of Primary Hit Candidates Using Western Blot Analysis and Quantitative Immunofluorescence Staining in 3D AD Models with High Aβ42/40 Ratios (#HReN and #A4H1)

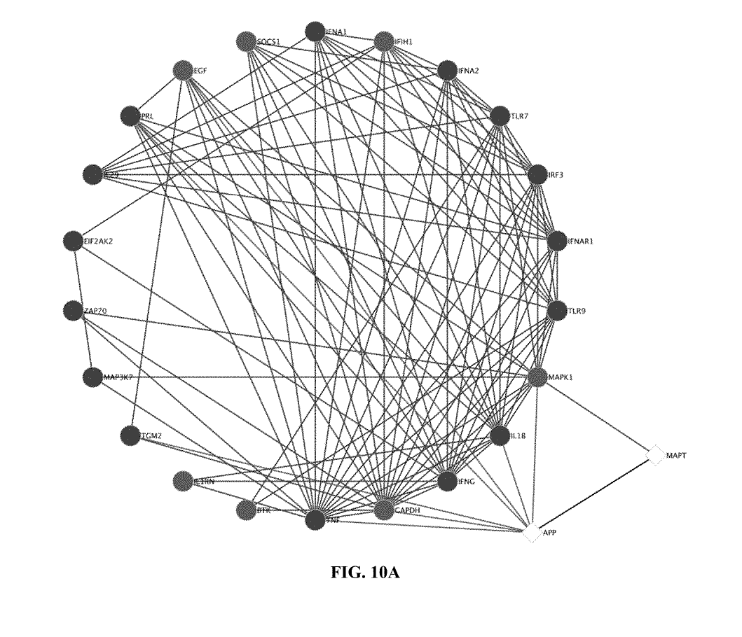

In addition to MSD Mesoscale ELISA shown in FIG. 8, quantitative Western blot and immunofluorescence analysis were used to validate candidate drugs. FIG. 9a shows Western blots further validating the impact of candidate drugs on p-tau species. Ebselen and leflunomide are compounds screened from original HCS screening of ˜24,00 biologically active/FDA-approved drug library. These compounds significantly decreased insoluble p-tau species (pSer396/Ser404, pThr181) in various concentrations (FIG. 9a). Moreover, quantitative immunofluorescence staining was used to analyze p-tau changes after treating these compounds. As shown in FIG. 9b, treatment with 5 μM leflunomide for 3 weeks robustly decreased p-tau (pSer396/Ser404) accumulation without affecting cellular viability and neurite networks.

### Example 6. Computational Modeling of RNAseq Data Reveal Possible Mechanisms Corresponding to Primary Screening Hits

The SMART framework disclosed herein can identify novel mechanisms underlying phenotypes of interest, e.g. inhibition of pTau accumulation and related pathways. Novel mechanisms identified in each round allows update on molecular signature and modification of compound ranking methods, thus generating iterative prediction-validations loops exploring different area of the searching space that might be flossed over with initial ranking strategy.

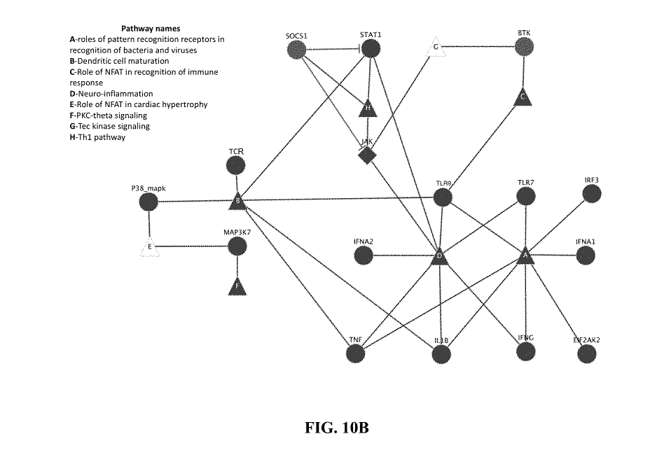

Given ebselen and leflunomide in FIG. 9, an unbiased whole genome RNAseq analysis was used to obtain transcriptomic profiles after the treatment of each compound and compare them separately to control conditions. For both treatments, a subset of genes and pathways show significant change (|log FC|>1.5) in the same direction over control condition. FIG. 10a shows a tightly-knit PPI subnetwork involving 15 down-regulated and 7 up-regulated genes after both compound treatments. These 22 genes have 102 PPI pairs among them, and there are 7 genes directly connected to APP (coding Aβ) or MAPT (coding Tau).

There are 12 down-regulated genes connected to 6 pathways, 5 of which are significantly down-regulated after treatment of both ebselen and leflunomide (FIG. 10b). It's worth noting that the enrichment of immune and inflammatory related pathway changes is consistent with the characteristics of the 3D cell model, as this system contains astrocytes, which is one of the brain innate immune cells. One of the only up-regulated genes, SOCS1, is a known suppressor for the activity of STAT-JAK pathway. Also, neuroinflammatory pathways are highly unregulated in high Abeta42/40 lines (D4 and H10) as compared to A5 (similar to GA2) (data not shown).

The thorough validation efforts using multiple human cell lines and various biochemistry and bioinformatics technologies (FIGS. 8 and 9) confirmed the ability of the SMART screening framework for identifying compounds for treating and/or preventing Alzheimer's Disease. The generation of customized RNAseq data help provide deeper insight of the similarity between the 3D cell system and AD pathology in vivo (FIG. 7), and also reveal clues for novel molecular mechanisms underlying various screening hits (FIG. 10). The generation and modeling of the RNAseq data shows the ability of the SMART framework to deal with transcriptome data generated from multiple platforms. Furthermore, FIG. 10b demonstrates that the bioinformatics methods for SMART shown herein can uncover novel mechanisms underlying pTau inhibition.

### REFERENCES CITED

1. Chong C R, Sullivan D J. New uses for old drugs. Nature. 2007 Aug. 9; 448(7154):645-646.
2. Walsh D P, Chang Y-T. Chemical Genetics. Chem Rev. 2006 Jun. 1; 106(6):2476-2530.
3. Diamandis P, Wildenhain J, Clarke I D, Sacher A G, Graham J, Bellows D S, Ling E K M, Ward R J, Jamieson L G, Tyers M, Dirks P B. Chemical genetics reveals a complex functional ground state of neural stem cells. Nat Chem Biol. 2007 May; 3(5):268-273. PMID: 17417631
4. Choi S H, Kim Y H, Hebisch M, Sliwinski C, Lee S, D/′Avanzo C, Chen H, Hooli B, Asselin C, Muffat J, Klee J B, Zhang C, Wainger B J, Peitz M, Kovacs D M, Woolf C J, Wagner S L, Tanzi R E, Kim D Y. A three-dimensional human neural cell culture model of Alzheimer/'s disease. Nature. 2014 Nov. 13; 515(7526):274-278.
5. Kim Y H, Choi S H, D'Avanzo C, Hebisch M, Sliwinski C, Bylykbashi E, Washicosky K J, Klee J B, Brustle O, Tanzi R E, Kim D Y. A 3D human neural cell culture system for modeling Alzheimer's disease. Nat Protoc. 2015 July; 10(7):985-1006.
6. Oddo S, Caccamo A, Shepherd J D, Murphy M P, Golde T E, Kayed R, Metherate R, Mattson M P, Akbari Y, LaFerla F M. Triple-Transgenic Model of Alzheimer's Disease with Plaques and Tangles: Intracellular Aβ and Synaptic Dysfunction. Neuron. 2003 Jul. 31; 39(3):409-421.
7. Lamb J, Crawford E D, Peck D, Modell J W, Blat I C, Wrobel M J, Lerner J, Brunet J-P, Subramanian A, Ross K N, Reich M, Hieronymus H, Wei G, Armstrong S A, Haggarty S J, Clemons P A, Wei R, Carr S A, Lander E S, Golub T R. The Connectivity Map: Using Gene-Expression Signatures to Connect Small Molecules, Genes, and Disease. Science. 2006 Sep. 29; 313(5795):1929.
8. Lamb J. The Connectivity Map: a new tool for biomedical research. Nat Rev Cancer. 2007 January; 7(1):54-60.
9. Library of Integrated Network-based Cellular Signatures (LINCS). [Internet]. Available from: https://commonfund.nih.gov/LINCS/10.
10. Duan Q, Reid S P, Clark N R, Wang Z, Fernandez N F, Rouillard A D, Readhead B, Tritsch S R, Hodos R, Hafner M, Niepel M, Sorger P K, Dudley J T, Bavari S, Panchal R G, Ma′ayan A. L1000CDS2: LINCS L1000 characteristic direction signatures search engine. Npj Syst Biol Appl. 2016 Aug. 4; 2:16015.
11. Jin G, Fu C, Zhao H, Cui K, Chang J, Wong S T C. A novel method of transcriptional response analysis to facilitate drug repositioning for cancer therapy. Cancer Res [Internet]. 2011 Nov. 22; Available from: http://cancerres.aacrjournals.org/content/early/2011/11/21/0008-5472.CAN-11-2333.abstract
12. Zhao H, Jin G, Cui K, Ren D, Liu T, Chen P, Wong S, Li F, Fan Y, Rodriguez A, Chang J, Wong S T. Novel Modeling of Cancer Cell Signaling Pathways Enables Systematic Drug Repositioning for Distinct Breast Cancer Metastases. Cancer Res. 2013 Oct. 14; 73(20):6149.
13. Jin G, Wong S T C. Toward better drug repositioning: prioritizing and integrating existing methods into efficient pipelines. Drug Discov Today. 2014 May; 19(5):637-644.
14. Azvolinsky A. Repurposing Existing Drugs for New Indications. The Scientist. 2017 Jan. 1;
15. Choi D S, Blanco E, Kim Y-S, Rodriguez A A, Zhao H, Huang T H-M, Chen C-L, Jin G, Landis M D, Burey L A, Qian W, Granados S M, Dave B, Wong H H, Ferrari M, Wong S T C, Chang J C. Chloroquine Eliminates Cancer Stem Cells Through Deregulation of Jak2 and DNMT1. STEM CELLS. 2014 Sep. 1; 32(9):2309-2323.
16. Chloroquine With Taxane Chemotherapy for Advanced or Metastatic Breast Cancer Patients Who Have Failed an Anthracycline (CAT) [Internet]. Available from: https://clinicaltrials.gov/ct2/show/NCT01446016
17. Zhang Y, Zhou X, Witt R M, Sabatini B L, Adjeroh D, Wong S T C. Dendritic spine detection using curvilinear structure detector and LDA classifier. NeuroImage. 2007 June; 36(2):346-360.
18. Fan J, Zhou X, Dy J G, Zhang Y, Wong S T C. An Automated Pipeline for Dendrite Spine Detection and Tracking of 3D Optical Microscopy Neuron Images of In Vivo Mouse Models. Neuroinformatics. 2009; 7(2):113-130.
19. Ofengeim D, Shi P, Miao B, Fan J, Xia X, Fan Y, Lipinski M M, Hashimoto T, Polydoro M, Yuan J, Wong S T C, Degterev A. Identification of Small Molecule Inhibitors of Neurite Loss Induced by Aβ peptide using High Content Screening. J Biol Chem. 2012 Mar. 16; 287(12):8714-8723.
20. Yin Z, Zhou X, Bakal C, Li F, Sun Y, Perrimon N, Wong S T. Using iterative cluster merging with improved gap statistics to perform online phenotype discovery in the context of high-throughput RNAi screens. BMC Bioinformatics. 2008; 9(1):1-20.
21. Yin Z, Zhou X, Sun Y, Wong S T C. Online phenotype discovery based on minimum classification error model. Pattern Recognit Comput Life Sci. 2009 April; 42(4):509-522.
22. Yin Z, Sadok A, Sailem H, McCarthy A, Xia X, Li F, Garcia M A, Evans L, Barr A R, Perrimon N, Marshall C J, Wong S T C, Bakal C. A screen for morphological complexity identifies regulators of switch-like transitions between discrete cell shapes. Nat Cell Biol. 2013 July; 15(7):860-871.
23. Yin Z, Sailem H, Sero J, Ardy R, Wong S T C, Bakal C. How cells explore shape space: A quantitative statistical perspective of cellular morphogenesis. BioEssays. 2014; 36(12):1195-1203.
24. De Bondt M, van den Essen A. Singular Hessians. J Algebra. 2004 Dec. 1; 282(1):195-204.
25. Chen K, Wang Y, Yang R. Hessian matrix based saddle point detection for granules segmentation in 2D image. J Electron China. 2008; 25(6):728-736.
26. Gu X-H, Xu L-J, Liu Z-Q, Wei B, Yang Y-J, Xu G-G, Yin X-P, Wang W. The flavonoid baicalein rescues synaptic plasticity and memory deficits in a mouse model of Alzheimer's disease. Behav Brain Res. 2016 Sep. 15; 311:309-321.
27. Corbett A, Pickett J, Burns A, Corcoran J, Dunnett S B, Edison P, Hagan J J, Holmes C, Jones E, Katona C, Kearns I, Kehoe P, Mudher A, Passmore A, Shepherd N, Walsh F, Ballard C. Drug repositioning for Alzheimer's disease. Nat Rev Drug Discov. 2012 November; 11(11):833-846.
28. Liberzon A, Subramanian A, Pinchback R, Thorvaldsdóttir H, Tamayo P, Mesirov J P. Molecular signatures database (MSigDB) 3.0. Bioinformatics. 2011 May 5; 27(12):1739-1740.
29. Vidovié D, Koleti A, Schürer S C. Large-scale integration of small molecule-induced genome-wide transcriptional responses, Kinome-wide binding affinities and cell-growth inhibition profiles reveal global trends characterizing systems-level drug action. Front Genet. 2014; 5:342.
30. Liu C, Su J, Yang F, Wei K, Ma J, Zhou X. Compound signature detection on LINCS L1000 big data. Mol Biosyst. 2015; 11(3):714-722.
31. Mootha V K, Lindgren C M, Eriksson K-F, Subramanian A, Sihag S, Lehar J, Puigserver P, Carlsson E, Ridderstrale M, Laurila E, Houstis N, Daly M J, Patterson N, Mesirov J P, Golub T R, Tamayo P, Spiegelman B, Lander E S, Hirschhorn J N, Altshuler D, Groop L C. PGC-1[alpha]-responsive genes involved in oxidative phosphorylation are coordinately downregulated in human diabetes. Nat Genet. 2003 July; 34(3):267-273.
32. Kanehisa M, Goto S. KEGG: Kyoto Encyclopedia of Genes and Genomes. Nucleic Acids Res. 2000 Jan. 1; 28(1):27-30.
33. Kanehisa M, Sato Y, Kawashima M, Furumichi M, Tanabe M. KEGG as a reference resource for gene and protein annotation. Nucleic Acids Res. 2015 Oct. 17; 44(D1):D457-D462.
34. Nishimura D. BioCarta. Biotech Softw Internet Rep [Internet]. 2001; 2. Available from: http://dx.doi.org/10.1089/152791601750294344
35. Biocarta pathways [Internet]. Available from: https://cgap.nci.nih.gov/Pathways/BioCarta Pathways
36. Milacic M, Haw R, Rothfels K, Wu G, Croft D, Hermjakob H, D'Eustachio P, Stein L. Annotating Cancer Variants and Anti-Cancer Therapeutics in Reactome. Cancers. 2012; 4(4).
37. Croft D, Mundo A F, Haw R, Milacic M, Weiser J, Wu G, Caudy M, Garapati P, Gillespie M, Kamdar M R, Jassal B, Jupe S, Matthews L, May B, Palatnik S, Rothfels K, Shamovsky V, Song H, Williams M, Birney E, Hermjakob H, Stein L, D'Eustachio P. The Reactome pathway knowledgebase. Nucleic Acids Res. 2013 Nov. 15; 42(D1):D472-D477.
38. Xie X, Lu J, Kulbokas E J, Golub T R, Mootha V, Lindblad-Toh K, Lander E S, Kellis M. Systematic discovery of regulatory motifs in human promoters and 3 [prime] UTRs by comparison of several mammals. Nature. 2005 Mar. 17; 434(7031):338-345.
39. KNÜPPEL R, DIETZE P, LEHNBERG W, FRECH K, WINGENDER E. TRANSFAC Retrieval Program: A Network Model Database of Eukaryotic Transcription Regulating Sequences and Proteins. J Comput Biol. 1994 Jan. 1; 1(3):191-198.
40. Ashburner M, Ball C A, Blake J A, Botstein D, Butler H, Cherry J M, Davis A P, Dolinski K, Dwight S S, Eppig J T, Harris M A, Hill D P, Issel-Tarver L, Kasarskis A, Lewis S, Matese J C, Richardson J E, Ringwald M, Rubin G M, Sherlock G. Gene Ontology: tool for the unification of biology. Nat Genet. 2000 May; 25(1):25-29.
41. Gene Ontology Consortium: going forward. Nucleic Acids Res. 2014 Nov. 26; 43(D1):D1049-D1056.
42. Liberzon A, Birger C, Thorvaldsdóttir H, Ghandi M, Mesirov J P, Tamayo P. The Molecular Signatures Database Hallmark Gene Set Collection. Cell Syst. 2015 Dec. 23; 1(6):417-425.
43. Iorio F, Bosotti R, Scacheri E, Belcastro V, Mithbaokar P, Ferriero R, Murino L, Tagliaferri R, Brunetti-Pierri N, Isacchi A, di Bernardo D. Discovery of drug mode of action and drug repositioning from transcriptional responses. Proc Natl Acad Sci. 2010 Aug. 17; 107(33):14621-14626.
44. Kuhn M, Szklarczyk D, Franceschini A, von Mering C, Jensen L J, Bork P. STITCH 3: zooming in on protein—chemical interactions. Nucleic Acids Res. 2011 Nov. 9; 40(D1):D876-D880.
45. Martin Y C, Kofron J L, Traphagen L M. Do Structurally Similar Molecules Have Similar Biological Activity? J Med Chem. 2002 Sep. 1; 45(19):4350-4358.
46. Online Mendelian Inheritance in Man, OMIM® [Internet]. McKusick-Nathans Institute of Genetic Medicine, Johns Hopkins University (Baltimore, Md.); Available from: https://omim.org/47.
47. Jensen L J, Saric J, Bork P. Literature mining for the biologist: from information retrieval to biological discovery. Nat Rev Genet. 2006 February; 7(2):119-129.
48. Šarić J, Jensen L J, Ouzounova R, Rojas I, Bork P. Extraction of regulatory gene/protein networks from Medline. Bioinformatics. 2005 Jul. 26; 22(6):645-650.
49. Bang-Jensen J, Gutin G. Directed graphs: Theory, Algorithms and Applications, 2nd edition. Springer; 2009.
50. You Z-H, Yin Z, Han K, Huang D-S, Zhou X. A semi-supervised learning approach to predict synthetic genetic interactions by combining functional and topological properties of functional gene network. BMC Bioinformatics. 2010; 11(1):343.
51. Barrat A, Barthelemy M, Pastor-Satorras R, Vespignani A. The architecture of complex weighted networks. Proc Natl Acad Sci USA [Internet]. 2004; 101. Available from: http://dx.doi.org/10.1073/pnas.0400087101
52. Stephenson K, Zelen M. Rethinking Centrality: Methods and Applications. Soc Netw [Internet]. 1989; 11. Available from: http://dx.doi.org/10.1016/0378-8733(89)90016-6
53. Brandes U, Fleischer D. Centrality measures based on current flow. Stacs 2005 Proc [Internet]. 2005; 3404. Available from: http://dx.doi.org/10.1007/978-3-540-31856-9_44
54. Cortes C, Vapnik V. Support-Vector Networks. Mach Learn. 1995; 20.
55. Chang C-. C, Lin C-. J. LIBSVM Libr Support Vector Mach. 2001.
56. Guyon I, Weston J, Barnhill S, Vapnik V. Gene Selection for Cancer Classification using Support Vector Machines. Mach Learn. 2002; 46(1):389-422.
57. Globally Harmonized System of Classification and Labelling of Chemicals (GHS), Rev. 6 [Internet]. United Nations; 2015. Available from: http://www.unece.org/trans/danger/publi/ghs/ghs_rev06/06files_e.html#c38156
58. Bhattacharya A, De R K. Divisive Correlation Clustering Algorithm (DCCA) for grouping of genes: detecting varying patterns in expression profiles. Bioinformatics [Internet]. 2008; 24. Available from: http://dx.doi.org/10.1093/bioinformatics/btn133
59. Lee J-H, Kim D G, Bae T J, Rho K, Kim J-T, Lee J-J, Jang Y, Kim B C, Park K M, Kim S. CDA: Combinatorial Drug Discovery Using Transcriptional Response Modules. PLOS ONE. 2012 Aug. 8; 7(8):e42573.
60. Huang L, Li F, Sheng J, Xia X, Ma J, Zhan M, Wong S T C. DrugComboRanker: drug combination discovery based on target network analysis. Bioinformatics. 2014 Jun. 11; 30(12):i228-i236.
61. Eisen M B, Spellman P T, Brown P O, Botstein D. Cluster analysis and display of genome-wide expression patterns. Proc Natl Acad Sci USA [Internet]. 1998; 95. Available from: http://dx.doi.org/10.1073/pnas.95.25.14863
62. Reich M, Liefeld T, Gould J, Lerner J, Tamayo P, Mesirov J P. GenePattern 2.0. Nat Genet. 2006 May; 38(5):500-501.
63. Opsahl T, Panzarasa P. Clustering in weighted networks. Soc Netw [Internet]. 2009; 31. Available from: http://dx.doi.org/10.1016/j.socnet.2009.02.002
64. Hinton G E, Osindero S, Teh Y-W. A Fast Learning Algorithm for Deep Belief Nets. Neural Comput. 2006 May 17; 18(7):1527-1554.
65. Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: A knowledge-based approach for interpreting genome-wide expression profiles. Proc Natl Acad Sci. 2005 Oct. 25; 102(43):15545-15550.
66. Gilks W R, Best N G, Tan K K C. Adaptive Rejection Metropolis Sampling within Gibbs Sampling. J R Stat Soc Ser C Appl Stat. 1995; 44(4):455-472.
67. Meyer R, Cai B, Perron F. Adaptive rejection Metropolis sampling using Lagrange interpolation polynomials of degree 2. Comput Stat Data Anal. 2008 Mar. 15; 52(7):3408-3423.
68. D'Avanzo C, Aronson J, Kim Y H, Choi S H, Tanzi R E, Kim D Y. Alzheimer's in 3D culture: Challenges and perspectives. BioEssays. 2015 Oct. 1; 37(10):1139-1148.
69. Xie W, Li X, Li C, Zhu W, Jankovic J, Le W. Proteasome inhibition modeling nigral neuron degeneration in Parkinson's disease. J Neurochem. 2010 Oct. 1; 115(1):188-199.
70. Dunkley P R, Jarvie P E, Robinson P J. A rapid Percoll gradient procedure for preparation of synaptosomes. Nat Protoc. 2008 October; 3(11):1718-1728.
71. Galli S, Lopes D M, Ammari R, Kopra J, Millar S E, Gibb A, Salinas P C. Deficient Wnt signalling triggers striatal synaptic degeneration and impaired motor behaviour in adult mice. Nat Commun. 2014 Oct. 16; 5:4992.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

We claim:

1. A method for screening for a modulator of a target protein, comprising:
contacting a cell with at least one primary candidate agent;
identifying the at least one primary candidate agent that modulates the target protein;
obtaining publicly available large transcriptomic profiles of cellular responses to the at least one primary candidate agent;
performing a first iteration to extract gene expression signatures for the at least one primary candidate agent;
ranking all secondary candidate agents from the publicly available large transcriptomic profiles of cellular responses based on a similarity score of the transcriptomic profile to the at least one primary candidate agent;
selecting the modulator of a target protein from the secondary candidate agents when the similarity score is above a determined threshold.

2. The method of claim 1, wherein the target protein is tau.

3. The method of claim 1, wherein the modulator affects tau phosphorylation.

4. The method of claim 1, wherein the similarity score of the transcriptomic profile is measured by a cMAP algorithm.

5. The method of claim 1, wherein at least one additional iteration is performed, wherein the modulator of a target protein is added back to the list of primary candidate agents, and new modulators of the target protein are obtained by repeating the screening process.

6. The method of claim 1, wherein the gene expression signatures include whole genome transcriptomic profiles.

7. The method of claim 1, wherein the gene expression signatures include transcriptomic profiles for selected gene sets.

8. A computer implemented method of selecting viable target agents having a predicted drug interaction response in a patient, the method comprising:
a computer processor connected to computerized memory storing computer implemented instructions configured to iteratively repeat the following steps until converging on a final set of viable target agents:
retrieving search results from a database stored in the memory and accessible by the processor, wherein said search results identify a first set of primary candidate agents;
ranking the primary candidate agents in the first set according to pre-established criteria stored in the memory;
storing in the memory a search set of molecular traits for a selected set of laboratory validated agents selected from the ranked primary candidate agents;
using the search set of molecular traits to search the database for additional sets of secondary candidate agents exhibiting the molecular traits.

9. The computer implemented method of claim 8, wherein the molecular traits comprise a molecular signature, a transcriptomic profile, or a phenotypical response.

10. The computer implemented method of claim 8, wherein the computer implemented instructions are further configured to modulate the molecular signature data in the search set to tune the search set to a preferred phenotype.

11. A computer implemented method of identifying a set of target agents capable of completing selected biochemical tasks in a drug interaction process, the method comprising:
a computer processor connected to computerized memory storing computer implemented instructions configured to iteratively repeat the following steps until converging on a final set of viable target agents;
performing an electronic search of at least one database stored in the memory and accessible by the processor, wherein said search results identify a set of primary candidate agents;
extracting a signature for a target phenotype from each of said primary candidate agents;
compiling an expression profile in regard to the target phenotype for each of primary candidate agents;
ranking the primary candidate agents in the set according to pre-established criteria stored in the memory;
storing in the memory a search set of molecular traits for a selected set of laboratory validated agents selected from the ranked primary candidate agents;
refining respective signatures for a target phenotype in regard to the laboratory validated agents and creating an updated search set of molecular traits; and
using the search set of molecular traits to search the database for additional sets of primary target agent candidates exhibiting the molecular traits.

12. The computer implemented method of claim 11, wherein extracting the signature comprises transforming transcriptomic data for the primary candidate agents into a series of enrichment scores.

13. The computer implemented method of claim 12, wherein the enrichment scores comprise compressed representations of the transcriptomic data.

14. The computer implemented method of claim 11, wherein the ranking comprises summarizing the expression signatures and comparing to control conditions.

15. The computer implemented method of claim 11, wherein the ranking comprises generating a combined score incorporating similarities between perturbation profiles and chemical properties for each primary candidate agent and comparing the combined score.
